(19) United States
(12) Patent Application Publication
(10) Pub. No.: US 2013/0071933 A1
(43) Pub. Date: Mar. 21, 2013

(54) CITRUS SHOOT REGENERATION COMPOSITIONS, METHODS, AND SYSTEMS

(71) Applicant: Ian S. Curtis, Urbana, IL (US)

(72) Inventor: Ian S. Curtis, Urbana, IL (US)

(73) Assignee: THE TEXAS A&M UNIVERSITY SYSTEM, College Station, TX (US)

(21) Appl. No.: 13/618,548

(22) Filed: Sep. 14, 2012

Related U.S. Application Data
(60) Provisional application No. 61/536,844, filed on Sep. 20, 2011.

(51) Int. Cl. C12N 5/04

(52) U.S. Cl. 435/430; 435/431

(57) ABSTRACT

The present disclosure relates, according to some embodiments, to citrus shoot regeneration compositions, methods, and systems. For example, citrus shoot regeneration compositions (e.g., culture media) may comprise one or more non-ionic surfactants. Methods may comprise preparing and using these compositions in some embodiments. Regeneration systems may comprise, in some embodiments, one or more of these compositions and/or one or more citrus explants.

### CROSS-REFERENCE TO RELATED APPLICATIONS

This application claims the benefit of U.S. Provisional Application No. 61/536,844 filed on Sep. 20, 2011, the entire contents of which are hereby incorporated by reference.

### FIELD OF THE DISCLOSURE

The present disclosure relates, in some embodiments, to citrus shoot regeneration compositions, methods, and systems.

### BACKGROUND OF THE DISCLOSURE

Sweet orange (Citrus sinensis) represents the most economically important citrus species with a projected world production estimated at 64 million MT in 2010. However, the rate of development of new and improved cultivars is slow due to a long juvenile period, high heterozygosity and/or sexual incompatibility factors. Gene transfer technologies such as Agrobacterium-mediated transformation, particle gun bombardment, and protoplast fusion may each offer an alternative approach for the transfer of gene traits into important germplasms. The potential of these approaches is encumbered, however, by existing methods directed to the culture of juvenile tissue, in particular, epicotyl segments as explants, because these methods undesirably take years to produce plants that can be evaluated for horticultural and commercial traits.

### SUMMARY

Accordingly, a need has arisen for an improved reliable and efficient system (e.g., tissue culture system) for the production of regenerated shoots. The present disclosure relates, according to some embodiments, to citrus shoot regeneration compositions, methods, and systems.

In one example embodiment, a method of regenerating citrus is provided, the method comprising providing an explant of citrus, contacting the explant with a culture media comprising a non-ionic surfactant, and cultivating the explant under conditions that permit explant growth and/or flowering in less than about 16 months. In some embodiments, the explant comprises a mature stem internodal explant. In some embodiments, the non-ionic surfactant is selected from the group consisting of a polyoxypropylene-polyoxyethylene block copolymer, a polyethylene glycol p-(1,1,3,3-tetramethylbutyl)-phenyl ether, and/or, a polyoxyethylene (20) sorbitan monolaurate, and combinations thereof. In particular sub-embodiments, the concentration of the polyoxypropylene-polyoxyethylene block copolymer is from about 0.0005% to about 0.05%. In other sub-embodiments, the concentration of the polyethylene glycol p-(1,1,3,3-tetramethylbutyl)-phenyl ether is from about 0.0005% to about 0.05%. In yet other sub-embodiments, the concentration of the polyoxyethylene (20) sorbitan monolaurate is from about 0.05% to about 0.5%. Methods according to the present disclosure may also comprise, in some embodiments, assessment of one or more horticultural traits of the regenerated plant. Methods according to the present disclosure may also comprise, in some embodiments, the step of transforming the explant with an exogenous nucleic acid prior to contacting the explant with a culture media comprising a non-ionic surfactant. In some embodiments, the explant of citrus is a mature sweet orange cv. Hamlin.

In one example embodiment, a culture media for regenerating citrus is provided, the composition comprising a non-ionic surfactant selected from the group consisting of a polyoxypropylene-polyoxyethylene block copolymer at a concentration of from about 0.0005% (w/v) to about 0.05% (w/v), a polyethylene glycol p-(1,1,3,3-tetramethylbutyl)-phenyl ether at a concentration of from about 0.0005% (w/v) to about 0.05% (w/v), a polyoxyethylene (20) sorbitan monolaurate at a concentration of from about 0.05% (w/v) to about 0.5% (w/v), one or more salts, one or more carbohydrates, one or more plant growth regulators, and one or more vitamins. In some embodiments, the culture media further comprises coconut water. In some embodiments, the culture media further comprises a gel material. In some embodiments, the citrus is a mature citrus cv. Hamlin.

In one embodiment, a system for regenerating citrus is provided, the system comprising a culture media comprising a non-ionic surfactant selected from the group consisting of a polyoxypropylene-polyoxyethylene block copolymer at a concentration of from about 0.0005% (w/v) to about 0.05% (w/v), a polyethylene glycol p-(1,1,3,3-tetramethylbutyl)-phenyl ether at a concentration of from about 0.0005% (w/v) to about 0.05% (w/v), a polyoxyethylene (20) sorbitan monolaurate at a concentration of from about 0.05% (w/v) to about 0.5% (w/v) and a citrus explant comprising a mature internodal stem segment. In some embodiments, the citrus explant is a sweet orange cv. Hamlin. In some embodiments, the citrus explant is a mature sweet orange cv. Hamlin.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

Some embodiments of the disclosure may be understood by referring, in part, to the present disclosure and the accompanying drawings, wherein:

FIG. 1A illustrates normal shoot development of explants on DBA3 medium containing 0.001% (w/v) Pluronic® F-68 according to a specific example embodiment of the disclosure (bar: 3 cm);

FIG. 1B illustrates early shoot development of explants cultured on DBA3 medium supplemented with Tween® 20 according to a specific example embodiment of the disclosure (bar: 0.5 cm);

FIG. 1C illustrates late shoot development of explants cultured on DBA3 medium supplemented with Tween® 20 according to a specific example embodiment of the disclosure (bar: 0.5 cm);

FIG. 1D illustrates tissue culture-derived shoots showing successful acclimatization and growth after grafting according to a specific example embodiment of the disclosure (bar: 5 cm); and

FIG. 1E illustrates flowering and early fruit set shown by a successful graft 14-16 months after transfer to the greenhouse according to a specific example embodiment of the disclosure (bar: 5 cm).

### DETAILED DESCRIPTION

The present disclosure relates, according to some embodiments, to citrus shoot regeneration compositions, methods, and systems. For example, the present disclosure relates to citrus shoot regeneration compositions (e.g., culture media) comprising one or more non-ionic surfactants, methods of preparing and using these compositions, and regeneration systems that include these compositions in some embodiments. A reliable shoot regeneration system for mature tissue of citrus may be of major importance to accelerate the evaluation of commercial traits. Three non-ionic surfactants were evaluated independently in terms of their affects on the growth and regeneration of mature internodal stem segments of sweet orange cv. Hamlin in culture (Examples 1-7, below). As illustrated in these Examples, growth and/or shoot development of explants may be influenced by type of surfactant added to the regeneration medium DBA3, its concentration and/or order of flush growth used for explant preparation. Supplementation of Pluronic® F-68 at 0.001% (w/v) to the medium was the superior treatment resulting in significantly higher fresh weight gain of explant, improved mean number of shoots per explant and the percentage of explants giving shoots (33.5% from first flush) and shoot yield was 2-fold higher compared to treatments without surfactant (17%). Triton® X-100 was the least responsive in terms of its affect on the growth and regeneration of stem segments but such shoots had a normal phenotype. Explants cultured on DBA3 medium containing Tween® 20 exhibited growth and shoot yield similar to treatments without surfactant, but at concentrations 0.01-0.5% (v/v), the shoots became vitrified and failed to graft successfully in vivo. Growth and shoot yield of explants showed a general decline between flushes especially from second and third harvests. Shoots derived from stem segments which were cultured on media containing Pluronic® F-68 and no surfactant had a higher survival rate (70-80%, respectively) compared to treatments using Triton® X-100 at 0.001-0.1% (v/v) (33% survival). All acclimatized grafts exhibited typical mature wood characteristics and flowered 14-16 months after transfer to the greenhouse.

Attempts to reduce juvenility in citrus have succeeded by overexpressing flowering meristem identity genes such as the Arabidopsis LEAFY and APETALA genes in juvenile explants. However, such transformants exhibited pleiotropic effects, resulting in abnormal plant development. In a separate study, grafting adult buds onto juvenile rootstocks resulted in invigoration of mature tissue for transformation studies, resulting in flowering and fruit set approximately one year after regeneration. Finally, early flowering mutants (e.g., precocious trifoliate orange (Poncirus trifoliate (L.). Raf) with a 1-2 year juvenile period) may serve as useful models for evaluating genes in flower and fruit development in citrus. However, a regeneration system for mature stem segments may also bypass this juvenile phase and so accelerate the evaluation process of tissue culture-derived events according to some embodiments for the present disclosure.

Successful regeneration and transformation of mature internodal stem segments in citrus may be achieved using sweet orange cv. Pineapple in some embodiments. Other (e.g., more commercially important) cultivars may be induced to regenerate shoots from mature stem segments such as cv. Pera. Despite the genetic base of sweet oranges being relatively narrow, it is clear, that the media components required to promote optimal shoot production from mature tissue is quite diverse. Apart from the instant disclosure, the efficiency of regenerating shoots from commercially important mature tissues of citrus is relatively low compared to juvenile material.

Non-ionic, copolymer surfactants may possess cell protecting and growth stimulatory properties. In terms of plant cultures, there is now a growing list of materials and species, from protoplasts through to differentiated tissues and organs, which have demonstrated improvements in growth by the supplementation of media with low concentrations of copolymer surfactants. Several non-ionic surfactants have been tested in plant cultures and can be separated by their hydrophilic-hydrophobic balance (HLB) number. Surfactants such as Pluronic® F-68 (Poloxamer 118), has a high HLB number (29.0). Without limiting any particular embodiment to any specific mechanism of action, a chemical with a high HLB number may have a low capability to interact with the lipid fraction of cell membranes. However, in terms of its affect in stimulating growth and differentiation of cultured explants, it may be beneficial to a wide range of species, especially semi-woody plants, such as Woody Nightshade (Solanum dulcamara), Jute (Corchorus capsularis), Chrysanthemum and root crops such as cassava. Again, without limiting any particular embodiment to any specific mechanism of action, surfactants with a low HLB number, may have a higher capacity to interact with the lipid fraction of the cell membrane. Surfactants with a low HLB number have also demonstrated beneficial affects in plant cultures. For example, the non-ionic surfactants, Tween® 20 (HLB number, 16.7) and Triton X-100 (HLB, 13.5) were able to promote shoot regeneration of jute cotyledons. Accordingly, inclusion of non-ionic surfactants in the culture media may benefit the growth and regeneration of other woody plants such as citrus in some embodiments.

### Compositions

The present disclosure relates to citrus shoot regeneration compositions in some embodiments. Compositions (e.g., culture media) comprising one or more non-ionic surfactants may be, in some embodiments, effective and/or useful for growth and regeneration of mature stem tissues of citrus (e.g., mature stem internodal explants of sweet orange cv. Hamlin). In some embodiments, non-ionic surfactants may be utilized in improving the regeneration of mature tissue, which may have an important impact in producing shoot material without the juvenile phase and so allow earlier assessment of horticultural traits.

A surfactant, according to some embodiments of the disclosure, may comprise a non-ionic surfactant and/or a polysorbate surfactant. A non-ionic surfactant may comprise, for example, a polyoxypropylene-polyoxyethylene block copolymer (e.g., Pluronic® F-68) and/or a polyethylene glycol p-(1,1,3,3-tetramethylbutyl)-phenyl ether (e.g., Triton® X-100). A polysorbate surfactant may comprise polyoxyethylene (20) sorbitan monolaurate (e.g., Tween® 20). Any grade and/or purity of surfactant known to one of skill in the art may be used. In one embodiment, one or more tissue culture grade surfactant(s) is/are used. In one embodiment, one or more highly pure surfactant(s) is/are used. In this embodiment, minimal surfactant impurities are present. The one or more surfactant(s) may be about 90% to about 100% pure. The one or more surfactant(s) may be about 95% to about 100% pure. The one or more surfactant(s) may be about 90%, about 91%, about 92%, about 93%, about 94%, about 95%, about 96%, about 97%, about 98%, about 99%, or about 100% pure. In one embodiment, one or more surfactant(s) is/are used that are both tissue culture grade and highly pure.

According to some embodiments, the concentration of a surfactant may be any suitable concentration as assessed by, for example, the capacity to induce growth (e.g., weight gain) and/or shoot development in mature explants. Surfactant concentration may be from about 0.0001% (w/v) to about 1.0% (w/v). For example, surfactant concentration (e.g., total surfactant concentration and/or concentration of each surfactant present) may be from about 0.0005% (w/v) to about 0.005% (w/v), from about 0.001% (w/v) to about 0.01% (w/v), from about 0.005% (w/v) to about 0.05% (w/v), from about 0.01% (w/v) to about 0.1% (w/v), from about 0.05% (w/v) to about 0.5% (w/v), from about 0.1% (w/v) to about 0.5% (w/v), and/or from about 0.1% (w/v) to about 1.0% (w/v). A composition (e.g., a culture media) may comprise, in some embodiments, a surfactant selected from a polyoxypropylene-polyoxyethylene block copolymer (e.g., Pluronic® F-68) at a concentration of from about 0.0005% (w/v) to about 0.05% (w/v), a polyethylene glycol p-(1,1,3,3-tetramethylbutyl)-phenyl ether (e.g., Triton® X-100) at a concentration of from about 0.0005% (w/v) to about 0.05% (w/v), and/or a polyoxyethylene (20) sorbitan monolaurate (e.g., Tween® 20) at a concentration of from about 0.05% (w/v) to about 0.5% (w/v).

According to some embodiments, a composition (e.g., a culture media) may comprise one or more salts (e.g., Murashige and Skoog salts and/or a potassium salt), one or more carbohydrates (e.g., sucrose, reduced sugars, a water-soluble fraction of malted barley), one or more plant growth regulators (e.g., an auxin and/or a cytokinin), and/or one or more vitamins (e.g., Gamborg B5 vitamins). A composition (e.g., a culture media), in some embodiments, may comprise coconut water (e.g., de-proteinized coconut water) and/or a gel material (e.g., agar and/or Phytagel™). The pH of a composition may be adjusted as desired using a base.

### Systems

The present disclosure relates to citrus shoot regeneration systems in some embodiments. A system may comprise, according to some embodiments, a composition (e.g., a culture media) comprising a surfactant (e.g., a non-ionic surfactant) and/or a citrus explant (e.g., a mature citrus explant). In some embodiments, a citrus explant may be taken from any citrus species including, for example, sweet orange (Citrus sinensis). Other citrus species contemplated by the present disclosure include, but are not limited to, Citrus aurantifolia (key lime), Citrus maxima (pomelo), Citrus medica (citron), and Citrus reticulata (mandarin orange). In some embodiments, a citrus explant may be taken from any citrus hybrid, including, but not limited to, Citrus×aurantium (bitter orange), Citrus×latifolia (persian lime), Citrus×limon (lemon), Citrus×limonia (rangpur), Citrus×paradisi (grapefruit), and Citrus×tangerina (tangerine). According to some embodiments, the methods, compositions, and systems of the present disclosure may be used to regenerate shoots (e.g., from stem segments) of a variety selected from a Florida orange variety, a California orange variety, and/or a Texas orange variety. For example, shoots may be regenerated from cultivars selected from Hamlin, Midsweet, Valencia, Rhode Red Valencia, Parson Brown, Sunstar, Gardner, Temple, Dream Navel, Navel, Cara Cara (red) Navel, Jaffa, Pineapple, Washington Navel, Trovita, Fisher Navel, Fukumoto Navel, Cara Cara Navel, Late Australian Navel, Lane Late Navel, Shamouti (Jaffa), Midknight Valencia, Newhall, Marrs Navel Orange, N33 Navel Orange, Everhard Navel Orange, Parson Brown Orange, Joppa, and/or Olinda. Mature shoots may be regenerated from cultivars selected from Hamlin, Midsweet, Valencia, Rohde Red Valencia, Pineapple, Washington Navel, Marrs Navel, and/or N33 Navel Orange. Sweet orange varieties that may be transformed (e.g., by Agrobacterium-mediated transformation, particle gun bombardment, and/or protoplast fusion) and/or regenerated may include any suitable cultivar. Examples include, in some embodiments, sweet orange cv. Midsweet, sweet orange cv. Valencia, sweet orange cv. Rhode Red Valencia, sweet orange cv. Washington Navel, sweet orange cv. Marrs Navel Orange, sweet orange cv. N33 Navel Orange, sweet orange cv. Hamlin, sweet orange cv. Pineapple, and/or sweet orange cv. Pera. according to some embodiments. In some embodiments, a citrus explant may comprise any part of a juvenile and/or mature plant. For example, an explant may comprise any organ, tissue, and/or cell type. Explants may comprise, according to some embodiments, at least a portion of a stem, a leaf, and/or a root. Optionally, an explant may comprise one or more internodal segments (e.g., mature internodal stem segments).

### Methods

The present disclosure relates, according to some embodiments, to citrus shoot regeneration methods. A method may comprise, for example, contacting an explant (e.g., a mature citrus explant) with a composition (e.g., a culture media) comprising at least one surfactant (e.g., at least one non-ionic surfactant). A non-ionic surfactant may comprise, for example, a polyoxypropylene-polyoxyethylene block copolymer (e.g., Pluronic® F-68), a polyethylene glycol p-(1,1,3,3-tetramethylbutyl)-phenyl ether (e.g., Triton® X-100), and/or, a polyoxyethylene (20) sorbitan monolaurate (e.g., Tween® 20). In some embodiments, a method may comprise cultivation of an explant under conditions that permit regeneration of tissue (e.g., shoot tissue) substantially without or without a juvenile stage. A method may comprise grafting a regenerated plant or explant onto a shoot and/or rootstock of another plant. Grafted plants and/or explants may flower less than about 10 months after grafting, less than about 12 months after grafting, less than about 14 months after grafting, less than about 16 months after grafting, less than about 18 months after grafting, and/or less than about 20 months after grafting. A method may comprise, in some embodiments, transforming an explant (e.g., prior to contact with culture media comprising a surfactant). A method may comprise, according to some embodiments, use of tissue (e.g. citrus tissue) of any suitable age, including, but not limited to, juvenile tissue (immature tissue), mature tissue, and combinations thereof. The maturity of the tissue may be determined using any method known to one of skill in the art and is often based on the physiology and biochemistry of the material. Juvenile tissue may be tissue or material from a tree or plant which has not flowered and/or borne fruit. Mature tissue may be tissue or material from a tree or plant which has flowered and/or borne fruit. Mature tissue may be tissue or material from a tree or plant which would have flowered and/or borne fruit but for the presence and/or absence of a genetic trait. Mature tissue may be tissue or material from a tree or plant which would have flowered and/or borne fruit but for the presence and/or absence of a physiological trait. Mature tissue may be tissue or material from a tree or plant which would have flowered and/or borne fruit but for the presence and/or absence of an environmental condition. The time to maturity may vary among species. For example, in sweet orange, maturity requires about 12-14 years.

As will be understood by those skilled in the art who have the benefit of the instant disclosure, other equivalent or alternative compositions, devices, methods, and systems for citrus (e.g., mature citrus) regeneration can be envisioned without departing from the description contained herein. For example, compositions, methods, and systems are disclosed herein with identifiable components (e.g., steps, materials, compositions, articles, apparatus). In some embodiments, the disclosed components may be combined with additional components as desired and/or required. In some embodiments, the disclosed components may be exclusive of any other components as desired and/or required. Accordingly, the manner of carrying out the disclosure as shown and described is to be construed as illustrative only.

Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Persons skilled in the art may make various changes in methods of preparing and using a composition, device, and/or system of the disclosure. For example, a composition, method, and/or system may be prepared and or used as appropriate for use with juvenile and/or mature citrus (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value+/−about 10%, depicted value+/−about 50%, depicted value+/−about 100%) and/or a range endpoint. With respect to the former, a value of 50 depicted in an example, table, and/or drawing may form the basis of a range of, for example, about 45 to about 55, about 25 to about 100, and/or about 0 to about 100.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

### EXAMPLES

Some specific example embodiments of the disclosure may be illustrated by one or more of the examples provided herein.

### Example 1

### Plant Material and Establishment of In Vitro Cultures

Mature budwood collected from C. sinensis cv. Hamlin from virus-free greenhouse material were grafted onto seedlings of rough lemon (C. jambhiri Lush.) and maintained in a greenhouse (20-27° C.). The developing shoots were allowed to grow as one, two and three flushes, each used separately for experimental studies. Shoots (approx. 20 cm in length) were pruned from each plant and used for explant preparation. The stems were stripped of their thorns and leaves, surface-sterilized in 70% ethanol (1 min), disinfected for 20 min in 20% (v/v) domestic bleach solution containing 0.2% (v/v) Tween® 20 and washed five times with sterile distilled water. Internodal stem explants were cut transversely into 1 cm long segments and placed horizontally onto the culture media with the cut ends submerged (approx. 2 mm) into the medium. Explants were cultured on DBA3 medium (Murashige and Skoog (MS, 1962) salt mixture (bio-WORLD, Dublin, Ohio) supplemented with 20 mg l⁻¹ K₂HPO₄, 2.5% (w/v) sucrose, 0.1 mg l⁻¹ 2,4-dichlorophenoxyacetic acid (2,4-D), 3 mg l⁻¹ 6-benzyladenine (BA), 1.5 g l⁻¹ Bacto malt extract, Gamborg B5 vitamins (Gamborg et al. 1968), 0.02% (v/v) de-proteinized coconut water (Sigma-Aldrich) and 0.3% (w/v) Phytagel (bio-WORLD), pH adjusted to 5.8 using 1M KOH) (Deng et al. 1992), supplemented with freshly prepared Pluronic® F-68, Tween® 20 or Triton® X-100 at a concentration of 0, 0.001, 0.01, 0.1, 0.5% (10 explants/dish; 3 replicates/treatment; 2 experiments). The non-ionic surfactants were dissolved in sterile-distilled water and then filter-sterilized before adding to the medium after autoclaving, along with filter-sterilized Gamborg B5 vitamins and de-proteinized coconut water. Cultures were maintained in the light (60 μm m⁻²s⁻¹ cool light fluorescent tubes) under a 16 h photoperiod at 28±2° C. and subcultured at 21-28 d intervals. After 10 weeks, the weight of each explant and the number of adventitious shoots was recorded. This experiment was repeated for each of the first three flushes of shoots to compare growth and shoot regeneration efficiencies.

### Example 2

### Grafting of Adventitious Shoots onto Rootstocks

Adventitious shoots (0.5 cm long) were excised from mature stem segments and grafted in vivo onto rough lemon seedlings. Briefly, rough lemon seedlings (approx. 25-30 cm in height) were decapitated (approx. 15 cm above ground) where the diameter of the stem equals the width of the shoot (2-3 mm). A 5 mm deep longitudinal cut is made into the cambium layer of the seedling into which the shoot is wedged and the grafted area sealed with Parafilm. The shoot is wounded, each side, by removing approx. 1 mm of the surface with a scalpel blade in the same plane as the exposed cambium layer to promote growth of the new shoot. The whole plant is covered with a clear plastic bag and maintained in the light (100 μm⁻²s⁻¹ cool light fluorescent tubes) under a 16 h photoperiod at 22-24° C. After approx. 21 d, the grafted shoot starts to grow and the bag is gradually opened to aid acclimatization. The plant is then transferred to the greenhouse approx. 28 d later and allowed to grow to maturity.

### Example 3

### Statistical Analysis

Data on fresh weights and shoot production were analyzed statistically using a two-way analysis of variance (ANOVA), and differences between the means were compared using Student-Newman-Keuls test (P<0.05 was considered to be significantly different) by applying the statistical software SAS 9.1 (SAS Institute Inc., Cary, N.C., USA).

### Example 4

### Results: Fresh Weight Gain and Shoot Regeneration from Mature Stem Segments of Sweet Orange Cultured on DBA3 Medium Supplemented with Pluronic® F-68

Data for all treatments were combined, as there were no significant differences between the two experiments. The growth responses of mature stem segments from fourth and fifth flushes derived from trees grown under greenhouse conditions were not evaluated due to the severe contamination from within the tissue compared to explants prepared from earlier flushes (percentage explants contaminated from 780 explants: first flush, 3%; second, 3%; third, 5%; fourth, 62%; fifth, 72%). Such differences in contamination levels of mature stem segments may reflect the changes in climate conditions, such as relative humidity, within the greenhouse where mother trees are grown for the production of explant material. In this particular case, it was noted that the fourth and fifth flushes of growth occurred when there was a higher relative humidity (70-80%) compared to the earlier flushes (40-50%) in the greenhouse.

Fresh weight gain and shoot regeneration from mature internodal stem segments of sweet orange at 10 weeks of culture was influenced by the type of surfactant supplemented to the media, its concentration and the order when shoot material was harvested from mother plants for preparing explants (flush growth) (Table 1). For mature stem segments, supplementation of DBA3 medium with 0.001-0.01% (w/v) Pluronic® F-68 significantly (P<0.05) increased (389±17 mg f. wt.) mean fresh weight gain of cultures, compared to explants cultured on DBA3 with no surfactant (243±16 mg f. wt.; Table 1) from the first flush of growth. Explants cultured on higher concentrations (0.1-0.5%) (w/v) of Pluronic® F-68 showed no significant difference in terms of mean fresh weight compared to the control. The increased fresh weight of explants cultured on DBA3 with 0.001% (w/v) Pluronic® F-68 coincided with a significantly (P<0.05) increased (0.72±14) mean number of shoots per explant compared to the control (0.22±0.07) from the first flush of growth. In contrast, mature stem segments cultured on DBA3 medium supplemented with 0.01 (w/v) Pluronic® F-68 (0.33±0.1), showed no significant difference in mean number of shoots per explant compared to the control, suggesting that the increased fresh weight gain was not due to organogenesis but a result of increased callus development. Furthermore, these differences in the response of mature stem segments cultured on DBA3 with 0.001% and 0.01 (w/v) Pluronic® F-68 were also observed in all three flushes of shoots used for explant preparation (Table 1). These observations support previous observations of growth stimulation by Pluronic® F-68, but there appears to be a real difference in the response of explants in terms of different species, and other plant genera. For example, epicotyl explants of C. depressa, cultured on SRBI medium, only showed a significant increase in fresh weight gain and number of shoots per explant when cultured on medium supplemented with 0.5% (w/v) Pluronic® F-68 and lower concentrations significantly reduced growth development. Furthermore, cotyledonary explants of C. depressa, failed to show a significant difference in fresh weight gain when cultured on medium containing 0.001-0.5% (w/v) Pluronic® F-68. Thus, there appears to be a marked interspecific variation associated with the responsiveness of explants to Pluronic® F-68 within citrus. Direct comparisons using the same explant type, cultured on a range of concentrations of Pluronic® F-68 may further support this claim.

Interspecific variation of explants in response to Pluronic® F-68 in culture has also been previously reported in Passiflora. For example, leaf segments of P. gibertii cultured on medium supplemented with 0.001% (w/v) Pluronic® F-68 promoted maximum fresh weight gain and number of explants regenerating shoots. In contrast, for P. mollisima, maximum fresh weight gain and maximum number of shoots per explant, occurred with 0.1% (w/v) Pluronic® F-68. Marked genotypic variations of explants in response to Pluronic® F-68 have also been reported using a broad range of plant species. For example, transformed roots of Solanum dulcamara exhibited maximum growth when culture medium was supplemented with 0.01% (w/v) Pluronic® F-68, while that of leaf-derived callus from the same species, the maximum response occurred at 0.1% (w/v). In the case of Chrysanthemum, significant increases in biomass of cultured leaf explants occurred when explants were exposed to a wider range from 0.001-0.1% (w/v) Pluronic® F-68. In contrast, for Jute, the addition of culture medium with 0.5% (w/v) Pluronic® F-68 was maximal for shoot regeneration from cotyledonary explants, coupled, with maximal explant biomass. Pluronic® F-68 has also been shown to improve the growth and regeneration of nodal explants of cassava. In this study, maximum shoot regeneration and fresh weight gain occurred when culture medium was supplemented with 2% (w/v) Pluronic® F-68; many orders of magnitude higher than previous studies.

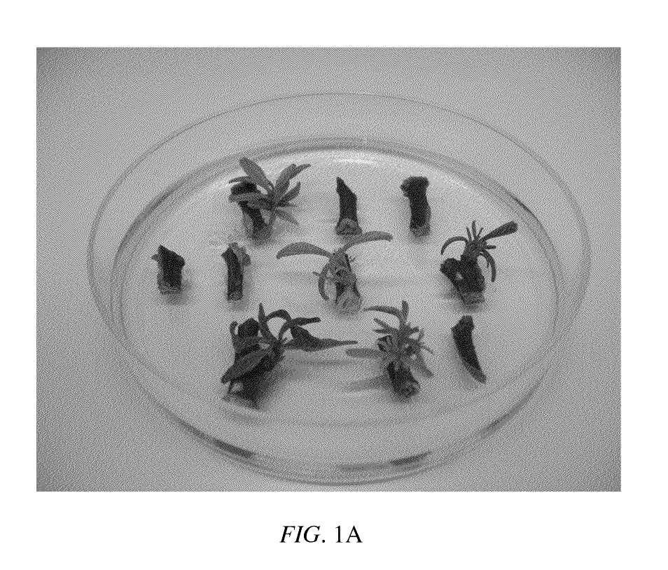

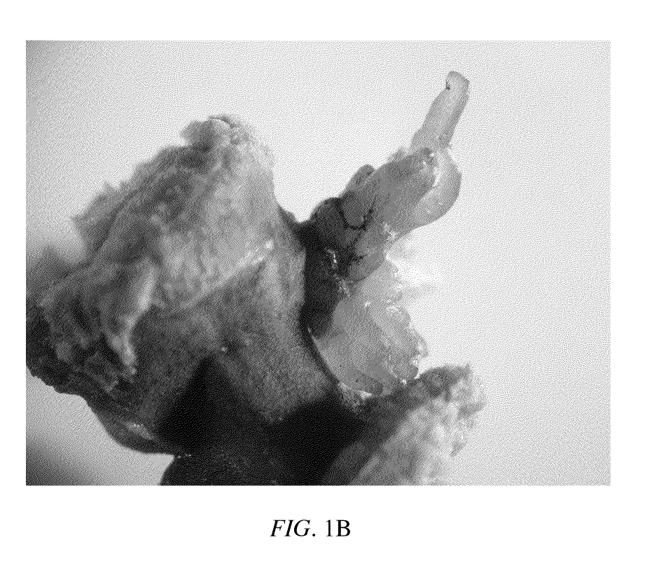

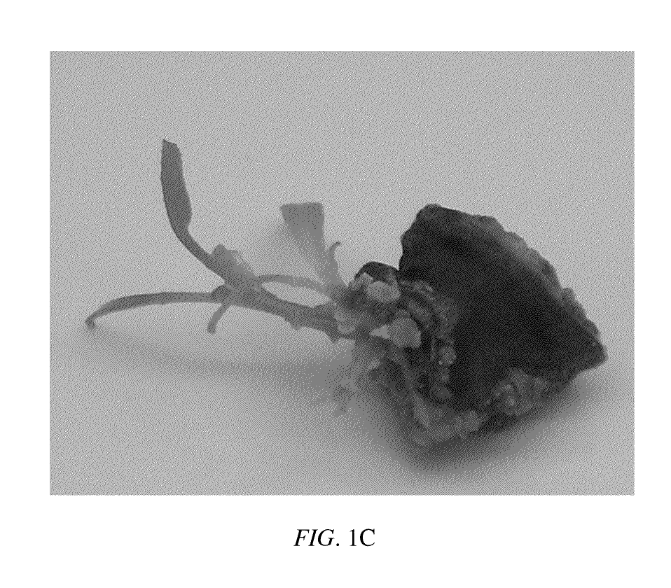

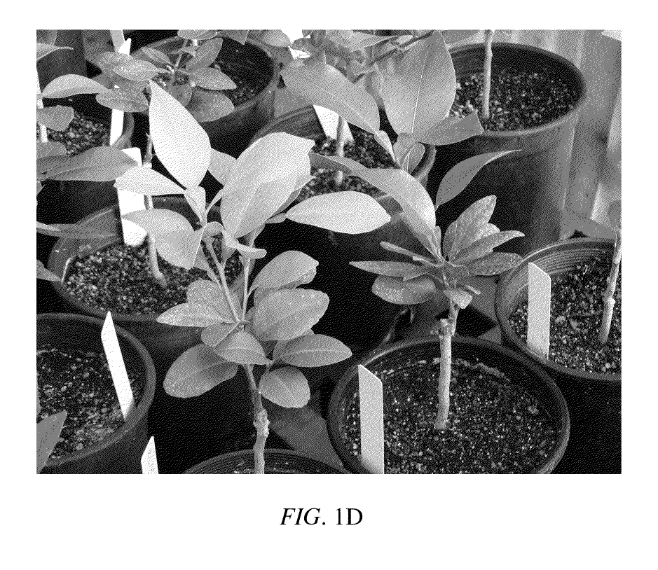

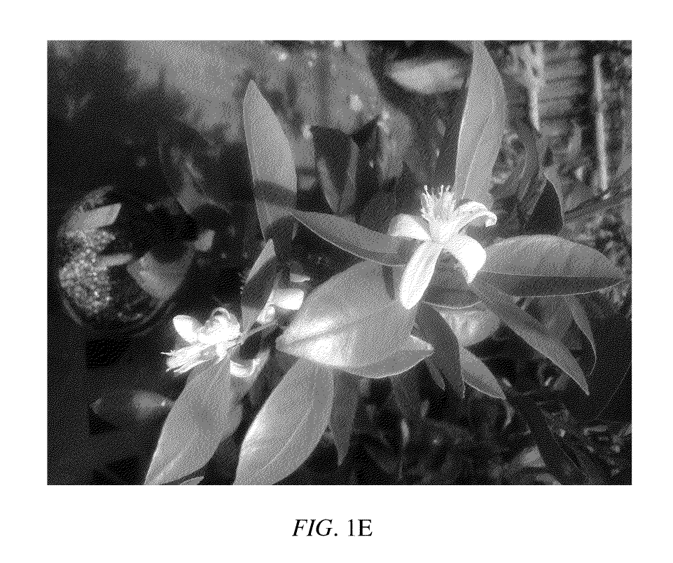

Throughout the three flushes of growth used for explant preparation, stem segments cultured on DBA3 supplemented with 0.001% (w/v) Pluronic® F-68 significantly (P<0.05) increased the mean number of shoots per explant compared to the control (Table 1; FIG. 1A). Indeed, the percentage of explants giving shoots in the first flush was almost double compared to the control (33.5%, Pluronic® F-68; 17%, control) (Table 2). Such a regeneration frequency for mature stem segments is extremely encouraging compared to the low frequencies reported previously in sweet orange. For example, the regeneration frequency of mature internodal stem segments of cv. Pineapple was 23% from the first flush and was as low as 5% from the third flush. Furthermore, in a separate study, stem internodal explants of cv. Hamlin that were inoculated with Agrobacterium, showed a regeneration frequency of only 15%. However, the regeneration efficiency of mature internodal stem segments, in all treatments, does reduce especially from flush 2 to flush 3. This reduction in the efficiency of mature stem explants to regenerate shoots from successive flushes has been reported previously in sweet orange cv. Pineapple and so maybe a common occurrence in some citrus.

In this example, the responses of cultured plant tissues to Pluronic® F-68 depended on the concentration used, the type of explant, and the species under investigation. The results on the response of stem internodal segments of sweet orange in terms of fresh weight gain and regeneration disclosed in this Example are no different. Without limiting any particular embodiment to a specific mechanism of interaction between Pluronic® F-68 and plant cells, low concentrations may increase the permeability of the plasma membrane and so facilitate the movement of culture nutrients into the cell. In support of this theory, was a study in Populus, which reported that a medium supplemented with Pluronic® F-68 enabled the growth regulator, thidiazuron, to be used at a 10-fold lower concentration than that normally used at promoting shoot regeneration. Higher concentrations (greater than 0.5% (w/v) of Pluronic® F-68 may be associated with (e.g., cause) detrimental, irreversible changes to the plasma membrane and so impair cell growth. In the present Example examining mature stem segments of sweet orange, it appears that at concentrations higher than 0.01% (w/v) Pluronic® F-68, the growth promotory effects of the surfactant becomes non significant compared to control.

TABLE 1
Effect on mature internodal stem segments of sweet orange cv. Hamlin in response to different
surfactants in culture, evaluated in terms of fresh weight and shoot regeneration

| | Flush 1 | | Flush 2 | | Flush 3 | |
|---|---|---|---|---|---|---|
| | | Mean | | Mean | | Mean |
| | | number of | | number of | | number of |
| Treatment/ | Mean fresh | shoots per | Mean fresh | shoots per | Mean fresh | shoots per |
| Concentration | weight (mg) | explant | weight (mg) | explant | weight (mg) | explant |
| Control | 243 ± 16 | 0.22 ± 0.07 | 269 ± 19 | 0.15 ± 0.05 | 148 ± 8 | 0.08 ± 0.04 |
| Pl—0.001 | 389 ± 17* | 0.72 ± 0.14* | 449 ± 20* | 0.43 ± 0.11* | 226 ± 9* | 0.23 ± 0.08* |
| Pl—0.01 | 428 ± 21* | 0.33 ± 0.10 | 435 ± 20* | 0.18 ± 0.07 | 248 ± 10* | 0.12 ± 0.05 |
| Pl—0.1 | 228 ± 11 | 0.26 ± 0.08 | 239 ± 13 | 0.25 ± 0.07 | 154 ± 7 | 0.10 ± 0.05 |
| Pl—0.5 | 193 ± 11 | 0.25 ± 0.07 | 245 ± 16 | 0.20 ± 0.07 | 114 ± 6 | 0.08 ± 0.04 |
| Tr—0.001 | 347 ± 24* | 0.13 ± 0.06 | 309 ± 23* | 0.07 ± 0.03 | 135 ± 7 | 0.08 ± 0.04 |
| Tr—0.01 | 254 ± 13 | 0.18 ± 0.07 | 264 ± 14 | 0.10 ± 0.05 | 126 ± 6 | 0.02 ± 0.02 |
| Tr—0.1 | 153 ± 7 | 0.08 ± 0.04 | 158 ± 7 | 0.05 ± 0.03 | 89 ± 3 | 0.00 ± 0.00 |
| Tr—0.5 | 75 ± 3 | 0.00 ± 0.00 | 85 ± 4 | 0.00 ± 0.00 | 61 ± 8 | 0.00 ± 0.00 |
| Tw—0.001 | 273 ± 15 | 0.23 ± 0.08 | 240 ± 15 | 0.22 ± 0.07 | 193 ± 8* | 0.07 ± 0.03 |
| Tw—0.01 | 218 ± 12 | 0.25 ± 0.07 | 260 ± 13 | 0.20 ± 0.07 | 161 ± 7 | 0.05 ± 0.03 |
| Tw—0.1 | 269 ± 13 | 0.31 ± 0.08 | 302 ± 19* | 0.22 ± 0.07 | 155 ± 7 | 0.08 ± 0.04 |
| Tw—0.5 | 253 ± 12 | 0.18 ± 0.06 | 250 ± 13 | 0.17 ± 0.06 | 130 ± 6 | 0.05 ± 0.03 |

The results represent the mean ± standard error of the mean (SEM) of three replicate dishes (10 explants/dish) from two combined experiments after 10 weeks of culture (total of 60 explants/treatment)
Pl = Pluronic ® F-68, Tr = Triton ® X-100, Tw = Tween ® 20, Control = no surfactant
*Significantly higher compared to control treatment (P < 0.05)

TABLE 2
Effects of different surfactants in medium on organogenic
response and shoot yield of mature internodal stem
segments of sweet orange cv. Hamlin

| | Flush 1 | | Flush 2 | | Flush 3 | |
|---|---|---|---|---|---|---|
| | % | | % | | % | |
| | Organo- | Total | Organo- | Total | Organo- | Total |
| Treatment/ | genic | shoot | genic | shoot | genic | shoot |
| Concentration | explants | yield | explants | yield | explants | yield |
| Control | 17 | 13 | 13 | 9 | 8.5 | 5 |
| Pl—0.001 | 33.5 | 43 | 25 | 26 | 15 | 14 |
| Pl—0.01 | 20 | 20 | 13.5 | 11 | 10 | 7 |
| Pl—0.1 | 18.5 | 16 | 18.5 | 15 | 8.5 | 6 |
| Pl—0.5 | 20 | 15 | 15 | 12 | 8.5 | 5 |
| Tr—0.001 | 10 | 8 | 6.5 | 4 | 6.5 | 5 |
| Tr—0.01 | 13.5 | 11 | 6.5 | 6 | 1.5 | 1 |
| Tr—0.1 | 6.5 | 5 | 5 | 3 | 0 | 0 |
| Tr—0.5 | 0 | 0 | 0 | 0 | 0 | 0 |
| Tw—0.001 | 15 | 14 | 15 | 13 | 7 | 4 |
| Tw—0.01 | 18.5 | 15 | 13.5 | 12 | 5 | 3 |
| Tw—0.1 | 23 | 19 | 17 | 13 | 8.5 | 5 |
| Tw—0.5 | 15 | 11 | 13.5 | 10 | 5 | 3 |

The results represent three replicate dishes (10 explants/dish) from two combined experiments after 10 weeks of culture (total of 60 explants/treatment)
Pl = Pluronic ® F-68, Tr = Triton ® X-100, Tw = Tween ® 20, Control = no surfactant

### Example 5

### Fresh Weight Gain and Shoot Regeneration from Mature Stem Segments of Sweet Orange Cultured on DBA3 Medium Supplemented with Triton® X-100

Supplementation of DBA3 medium with 0.001% (v/v) Triton® X-100 significantly (P<0.05) increased mean fresh weight of mature stem segments from first flush (347±24 mg f. wt.) and second flush (309±23 mg f. wt.) growth compared to the control (first flush, 243±16 mg; second flush, 269±19 mg; Table 1). The increased fresh weight of stem explants on DBA3 with 0.001% (v/v) Triton® X-100 for the first two flushes could not be related to organogenesis, but due to increased callus growth, as the number of shoots per explant (Table 1) and the number of explants giving shoots (Table 2) were both lower compared to the control. In contrast, mature stem segments cultured on DBA3 medium supplemented with 0.1-0.5% (v/v) Triton® X-100 significantly (P<0.05) reduced fresh weight gain and number of shoots per explant (Table 1) and the number of shoots giving shoots (Table 2) compared to explants cultured on DBA3 without surfactant. These observations support similarly a previous study using cotyledonary explants of Jute, which showed that explants cultured on a medium supplemented with 0.001% (v/v) Triton® X-100 increased also the growth of such explants but, in this case, through organogenesis, due to increases in both the percentage of explants producing shoots and number of shoots per cotyledon (Khatun et al. 1993b). Furthermore, concentrations of Triton® X-100 above 0.001% (v/v) inhibited the growth and regeneration of Jute explants.

In terms of shoot development, mature internodal stem explants of sweet orange cultured on medium containing Triton® X-100, regenerated shoots of normal phenotype. In contrast, shoots derived from cotyledonary explants of Jute cultured on medium containing 0.001% Triton® X-100, became necrotic after 14-21 days following transfer to shoot elongation medium (Khatun et al. 1993b). Although the true mechanism of how Triton® X-100 interacts with plant cells is unclear, it is thought that the surfactant can more readily interact with the lipid component of plasma membranes, which at higher concentrations can cause membrane solubilization (Helenius and Simons 1975). This suggests that although Triton® X-100 could be beneficial in terms of growth of explants in citrus, its application should be used at a low concentration to avoid cell lysis and ultimately death of the plant tissue.

### Example 6

### Fresh Weight Gain and Shoot Regeneration from Mature Stem Segments of Sweet Orange Cultured on DBA3 Medium Supplemented with Tween® 20

By contrast to fresh weight gain with Pluronic® F-68 or Triton® X-100, there was no significant difference between the weight of explants cultured on DBA3 supplemented with Tween® 20 and stem segments cultured on medium without surfactant for all three flushes of growth (Table 1). In contrast, for Jute, the addition of culture medium with 0.001-0.01% (v/v) Tween® 20 significantly increased fresh weight gain of cotyledonary explants, but at 0.5% (v/v) the surfactant was inhibitory to growth (Khatun et al., 1993, Plant Cell Rep 13: 49-53). This observation suggests, as seen in studies using Pluronic® F-68, that different plant species and organs respond differently to a specific concentration of Tween® 20.

Likewise, the regeneration of mature stem segments of sweet orange was similar to the control for all concentrations of Tween® 20 and flushes of growth used for explant preparation (Table 1 and 2). However, in contrast to our results from using Pluronic® F-68 and Triton® X-100 in the culture medium which regenerated phenotypically-normal shoots, all shoots regenerated from mature stem segments on medium containing 0.01-0.5% (v/v) Tween® 20, were all vitrified (FIGS. 1B-C). In comparison, for Jute, the supplementation of 0.001-0.01% (v/v) Tween® 20 to a regeneration medium significantly increased the number of regenerants from cotyledonary explants, but at 0.5% (v/v) Tween® 20, shoot production was inhibited (Khatun et al., 1993, Plant Cell Rep 13: 49-53). Despite the number of studies using Tween® 20 in plant cell culture is limited, there is a clear difference in the response of mature stem segments of citrus and cotyledonary explants of Jute in terms of their responsiveness to different concentrations of the surfactant.

Tween® 20 has a relatively low HLB number (16.7), meaning the surfactant can readily interact with lipids in the plasma membrane of plant cells and so cause membrane disruption if used at too high concentrations. Our results show that if Tween® 20 is used at 0.01-0.5% (v/v), all shoots become vitrified, suggesting that the surfactant is causing damage to the cell membrane of these regenerants. Interestingly, the use of Triton® X-100, a surfactant with a lower HLB number (13.5) compared to Tween® 20, failed to produce abnormal shoots. One possible explanation, is that Tween® 20 has a subtle, yet unknown inhibitory affect on some developmental processes within the cell which maybe specific to citrus rather than other plant species (Lowe et al. 1993, Agro-Food-Ind Hi-Tech 4: 9-13).

### Example 7

### Development of Grafted Shoots

Shoots (approx. 1 cm in length) were excised from explants and grafted onto a rough lemon rootstock to allow growth and development. Five shoots were selected at random from each treatment/concentration of surfactant, except for treatment 0.5% (v/v) Triton® X-100 which failed to regenerate shoots, and the ability to acclimatize and grow into mature plants was observed. Shoots from explants cultured on DBA3 medium containing Pluronic® F-68 (14/20, 70% shoots survived; FIG. 1D) had a similar survival rate compared to regenerants from stems cultured in the absence of surfactant (4/5, 80% survival), following grafting. Vitrified shoots (FIGS. 1B-C) developing from stem segments cultured on DBA3 supplemented with Tween® 20, all failed to initiate growth following grafting. Shoots derived from explants cultured on Triton® X-100 at 0.001-0.1% (v/v) had a lower survival rate (5/15, 33%) compared to the Pluronic treatments and control. Following growth initiation and acclimatization, all grafted shoots exhibited few thorns and flowered 14-16 months from transfer to the greenhouse (FIG. 1E). This phenotype of growth and development is typical of a mature citrus tree as observed in previous studies on the tissue culture of mature internodal stem segments (Cervera et al., 1998, Transgenic Res 7: 51-59).

It is clear, for the first time, that the type of surfactant supplemented to the culture medium has a major affect on the success of grafting such shoots onto a rootstock. As discussed earlier, in terms of the response of cultured explants to surfactant in relation to fresh weight gain and regeneration, that the physio-chemical properties of a surfactant, or HLB number, is a critical factor in the success of producing shoots in citrus for commercial evaluation. Supplementation of medium with Pluronic® F-68 for the culture of mature internodal stem segments of sweet orange, improves regeneration of shoots, which are phenotypically-normal and are able to be grafted successfully onto a rootstock with high efficiency compared to the other surfactants studied. It seems therefore, that the high HLB number for Pluronic® F-68 (29.0), enables the surfactant to stimulate citrus cells to regenerate without causing disruption to the plasma membrane within the range tested (0.001-0.5% w/v) and so all the shoots develop without abnormality. The shoot regeneration efficiencies observed in treatments using Pluronic® F-68 has shown to have potential in improving organogenesis in a poorly responsive explant. This discovery could in the future be utilized in improving the transformation efficiency of this globally important crop.

What is claimed is:

1. A method of regenerating mature sweet orange, the method comprising:
providing an explant of mature sweet orange;
contacting the explant with a culture media comprising a non-ionic surfactant; and
cultivating the explant under conditions that permit explant growth and/or flowering in less than about 16 months.

2. A method according to claim 1, wherein the mature sweet orange is a variety selected from the group consisting of cv. Hamlin, cv. Pineapple, cv. Pera, cv. Midsweet, cv. Valencia, cv. Rohde Red Valencia, cv. Washington Navel, cv. Marrs Navel Orange, and cv. N33 Navel Orange.

3. A method according to claim 1, wherein the cultivating the explant further comprises cultivating the explant substantially with or without a juvenile stage.

4. A method according to claim 1, wherein the explant comprises a mature stem internodal explant.

5. A method according to claim 1, wherein the non-ionic surfactant is selected from the group consisting of a polyoxypropylene-polyoxyethylene block copolymer, a polyethylene glycol p-(1,1,3,3-tetramethylbutyl)-phenyl ether, and/or, a polyoxyethylene (20) sorbitan monolaurate, and combinations thereof.

6. A method according to claim 1, wherein the non-ionic surfactant comprises a polyoxypropylene-polyoxyethylene block copolymer at a concentration of from about 0.0005% (w/v) to about 0.05% (w/v).

7. A method according to claim 1, wherein the non-ionic surfactant comprises a polyethylene glycol p-(1,1,3,3-tetramethylbutyl)-phenyl ether at a concentration of from about 0.0005% (w/v) to about 0.05% (w/v).

8. A method according to claim 1, wherein the non-ionic surfactant comprises a polyoxyethylene (20) sorbitan monolaurate at a concentration of from about 0.05% (w/v) to about 0.5% (w/v).

9. A method according to claim 1 further comprising assessment of one or more horticultural traits of the regenerated plant.

10. A method according to claim 1 further comprising transforming the explant with an exogenous nucleic acid prior to contacting the explant with the culture media comprising the non-ionic surfactant.

11. A culture media for regenerating mature sweet orange, the composition comprising:
a non-ionic surfactant selected from the group consisting of a polyoxypropylene-polyoxyethylene block copolymer at a concentration of from about 0.0005% (w/v) to about 0.05% (w/v), a polyethylene glycol p-(1,1,3,3-tetramethylbutyl)-phenyl ether at a concentration of from about 0.0005% (w/v) to about 0.05% (w/v), a polyoxyethylene (20) sorbitan monolaurate at a concentration of from about 0.05% (w/v) to about 0.5% (w/v);
one or more salts;
one or more carbohydrates;
one or more plant growth regulators; and
one or more vitamins.

12. A culture media according to claim 11, wherein the mature sweet orange is a variety selected from the group consisting of cv. Hamlin, cv. Pineapple, cv. Pera, cv. Midsweet, cv. Valencia, cv. Rohde Red Valencia, cv. Washington Navel, cv. Mars Navel Orange, and cv. N33 Navel Orange.

13. A culture media according to claim 11 further comprising coconut water.

14. A culture media according to claim 11 further comprising a gel material.

15. A system for regenerating mature sweet orange, the system comprising:
a culture media comprising a non-ionic surfactant selected from the group consisting of a polyoxypropylene-polyoxyethylene block copolymer at a concentration of from about 0.0005% (w/v) to about 0.05% (w/v), a polyethylene glycol p-(1,1,3,3-tetramethylbutyl)-phenyl ether at a concentration of from about 0.0005% (w/v) to about 0.05% (w/v), a polyoxyethylene (20) sorbitan monolaurate at a concentration of from about 0.05% (w/v) to about 0.5% (w/v); and
a sweet orange explant comprising a mature internodal stem segment.

16. A system according to claim 15, wherein the mature sweet orange is a variety selected from the group consisting of cv. Hamlin, cv. Pineapple, cv. Pera, cv. Midsweet, cv. Valencia, cv. Rohde Red Valencia, cv. Washington Navel, cv. Marrs Navel Orange, and cv. N33 Navel Orange.
